(19) United States
(12) Patent Application Publication
(10) Pub. No.: US 2008/0262446 A1
(43) Pub. Date: Oct. 23, 2008

(54) Receptacle and Method for Disposing of Bodily Waste Materials

(76) Inventors: Gerard Ryder, New Ross (IE); Anne Houston, New Ross (IE)

Correspondence Address: THE WEBB LAW FIRM, P.C., 700 KOPPERS BUILDING, 436 SEVENTH AVENUE, PITTSBURGH, PA, 15219, US

(21) Appl. No.: 11/917,699

(22) PCT Filed: Jun. 13, 2006
(86) PCT No.: PCT/IE06/00064

§ 371 Date: Jul. 7, 2008

(30) Foreign Application Priority Data
Jun. 29, 2005 (IE) 2005/0438

(51) Int. Cl. A61F 5/445

(52) U.S. Cl. 604/317

(57) ABSTRACT

A disposable receptacle for bodily waste materials from a bodily waste discharge outlet of a collection bag having an elongate body with a top end and formed with an internal, bodily waste material-receiving chamber; an inlet opening formed by the body, proximate to, or at, the top end thereof, the body being adapted to receive within the inlet opening the bodily waste discharge outlet of the collection bag so as to allow bodily waste materials within the collection bag to be discharged into the bodily waste material-receiving chamber; and securing means for holding the bodily waste material-receiving chamber in position relative to the collection bag or a disposal unit. A simple yet effective device which may be easily used by a bag wearer/user which is easily disposed of by flushing. It may be used to dispose of waste from an artificial excretory opening collected in ileostomy or colostomy bags.

### FIELD OF THE INVENTION

The present invention relates to a receptacle and method for disposing of bodily waste materials. In particular the present invention concerns disposal of bodily waste materials which are collected from an artificial excretory opening.

Surgical construction of an artificial excretory opening generally takes place as an ostomy procedure. In particular, the present invention is useful to ostomy patients such as those who have had an ileostomy or colostomy.

### BACKGROUND TO THE INVENTION

Following surgery patients who have had a surgical construction of an artificial excretory opening such as ileostomy or colostomy patients use ileostomy/colostomy bags to collect bodily waste materials. These bodily waste materials include gases, liquids and solids. The waste material may be semi-solid faecal waste. It is desirable in any event to discharge the collected materials with minimal handling from the user.

These ileostomy/colostomy bags are intended for multiple use purposes and generally the surgical patient finds that they have to empty the collection bag many times during the day. A given collection bag is thus fitted with waste discharge outlet through which the waste materials collected from the artificial excretory opening can be discharged. It is not unusual to empty these bags between six and ten times in any given day. A collection bag will typically be worn for a number of consecutive days before being replaced with a new bag.

During the emptying procedure the patient or bag wearer generally has to squat down beside a toilet to allow direct emptying of the collection bag into the toilet. This is due in part to the typical positioning of the artificial excretory opening on the body. Often times the opening on the body is provided at or about the stomach area and often to one side of the body. It is not usually possible therefore for the bag wearer to easily directly empty the collected bodily waste material into a toilet if they are in a seated position on the toilet. A position to one side of the toilet is thus required.

Another option which some bag wearers use is to empty the collected bodily waste materials into a receptacle such as a plastic jug. The receptacle may then in turn be emptied into a toilet. Even with this method of emptying the bag it is difficult to use a toilet in a conventional fashion as if seated the user will likely have to stand up to empty the receptacle.

Generally therefore conventional procedures for emptying such collection bags are inconvenient.

This problem of inconvenience for the user has been identified. The inventors are aware of a number of proposals which seek to help a bag wearer dispose of bodily waste materials from the bag.

For example, UK patent application number GB 2,258,399 describes a plumbed device for emptying and cleaning a collection bag for excreta. The device has an inlet conduit, an outlet conduit and sealing means for connection with the opening of the bag in a substantially gas-tight manner. The device is formed by a number of plastic pipe portions which allowed the device to be connected to a water supply. The water supply accelerates drainage and affects cleaning of the bag. The outlet pipe sections are connected together to form a pipe of a length which can be located in the toilet under the U-trap thereof. In use the inlet section is connected to a water tap. While such a device may be suitable for use in a bag wearer's home, it is not generally suitable for convenient use when a bag wearer is on the move and may use different toilets during the day. The device is not portable in any convenient way. It will generally be fixed to a given toilet.

Another plumbing arrangement is described in U.S. Pat. No. 4,285,076. That patent describes a flushing apparatus for cleaning and flushing out ileostomy bags. It includes an arrangement including a funnel which is adapted to be fitted to a toilet. A flushing arrangement for the device is also provided. Again the device is not portable in any convenient way, as it will generally be fixed to a given toilet.

U.S. Pat. No. 6,695,825 discloses a portable, self-contained, powered unit designed to wash and clean the stoma and interior of drainable ostomy pouches. U.S. Pat. No. 5,671,485 discloses a device that can be fitted onto a toilet seat to support a colostomy bag. The device which is a metallic wire is placed on top of the toilet bowl and secured to the bowl through the use of brackets fastened over the rim. Other ostomy pouches, which may be disposed through toilets, are described in Canadian patent application no. 1,320,324 and U.S. Pat. No. 5,769,831. U.S. Pat. No. 4,930,942 describes a wrapping or enclosure into which flexible soft objects such as ostomy pouches can be placed. The material of the wrapping or enclosure becomes slimy on contact with water in the toilet bowl thus allowing for flushing. An inner and outer bag arrangement is described in the German patent application DE 19,519,069.

U.S. Pat. No. 5,938,647 to Welland Medical Limited discloses a biodegradable flushable ostomy bag liner. The liner is designed to be used in a two-bag ostomy bag system. The liner is adapted to fit into the outer bag and collect the waste material. When full the liner can be removed from the outer bag and disposed of by flushing in a toilet. The outer bag may have to be disposed of separately for example in a dustbin.

One of the drawbacks of this system is that the end user has to replace both the inner and the outer bag when the inner bag is full. This may prove difficult for people with a lack of dexterity.

One commercial toilet flushable pouch (similar to the device described in U.S. Pat. No. 5,938,647) is available from Welland Medical Limited in the UK. Their product sold under the trademark Flair Xtra™ is an inner liner pouch which is designed to fit within the bag worn by the user. The pouch is designed and arranged to collect the bodily waste materials and provide a barrier between those materials and the bag. None of the bodily waste materials therefore comes into direct contact with the inner of the bag. Additionally, the bodily waste materials can be removed by removing the inner pouch without contamination of the bag.

This means however that the inner pouch must be of a relatively complex design as it must be adapted to fit to the artificial opening of the body in the same way as the collection bag. This is necessary in order to avoid contamination of the collection bag with bodily waste materials.

### OBJECT OF THE INVENTION

It is an object of the present invention to provide a receptacle and method for disposing of bodily waste materials that overcomes the problems of the prior art above.

It is a further object of the present invention to provide a receptacle that is both portable and simple and convenient for the end user to employ.

### SUMMARY OF THE INVENTION

The present invention provides a receptacle and method for disposing of bodily waste materials.

In particular the present invention provides a disposable receptacle for receiving bodily waste materials from a bodily waste discharge outlet of a collection bag arranged to collect and retain bodily waste materials from a user's body, the receptacle comprising:
(i) an elongate body with a top end, a base end, an intermediate portion between the top end and the base end, and formed with an internal, bodily waste material-receiving chamber;
(ii) an inlet opening formed on the body, proximate to, or at, the top end thereof, the body being adapted to receive within (and through) the inlet opening the bodily waste discharge outlet of the collection bag so as to allow bodily waste materials within the collection bag to be discharged into the bodily waste material-receiving chamber; and
(iii) securing means for holding the bodily waste material-receiving chamber in position for discharge of the bodily waste materials into the bodily waste material-receiving chamber from the collection bag.

The present invention therefore provides a simple yet effective device which may be easily used by a bag wearer/user. It is easily disposed of also.

The securing means may be for securing the waste material receiving chamber:
(i) relative to the collection bag; or
(ii) relative to a disposal unit; or
(iii) relative to the collection bag and a disposal unit. For example there may be at least two securing means.

The securing means described above may enable the bodily waste material receiving chamber to be secured (and supported) in a usable position. This allows the end user to have both hands free to arrange the outlet of the collection bag in a discharge position.

Any form of such securing means may be provided including press-fit such as certain self-securing arrangements. The securing means for securing the waste material receiving chamber relative to the collection bag may be referred to as a “collection securing means” below. The securing means for securing the waste material receiving chamber relative to the disposal unit may be referred to as a “disposal unit securing means” below. Other manual attachment arrangements may also be provided. The securing means will also allow for ease of detaching the disposable receptacle from the bag or disposal unit. In this way the receptacle can be easily be removed from its (secured) position connected to the bag or disposal unit for disposal.

In one arrangement of the present invention it is desirable that the receptacle body is self-securing, for example to the collection bag. In this respect the receptacle body may be provided with adhesive for self-adhering to the collection bag. For example a peel-off tab may be provided over an adhesive patch on the receptacle. Removal of the peel-off tab exposes the adhesive. The adhesive can then be utilised to fulfil the securing function.

Other self-securing attachments may be used, such as any suitable type of fastener, such as hook and loop (e.g. Velcro™) or other snap fit or push fit attachments. Where two-part fasteners are employed then one part may be provided on the collection bag and the other on the receptacle. The user may be provided with the attachments for fitting as appropriate prior to use of the receptacle. If a fastener of any type is provided on the collection bag, for example to hold close a discharge outlet, that fastener may be employed to hold the receptacle to the collection bag.

The securing means may take the form of a hanging attachment which is provided to hold the receptacle in position relative to the collection bag. For example, the hanging attachment may be a loop which is arranged to hang from the collection bag. Any suitable hanging point may be employed. One suitable hanging point is a point of connection of the collection bag to the user's body. That point of connection is usually formed by two interconnecting pieces—a first piece on the body (about the bodily artificial excretory opening) and a second piece which is on the collection bag. The two pieces mate to form an exit conduit (usually sealed) to the collection bag. In such a case the hanging attachment can be hung off the mated connection. Generally the mated connection provides a secure attachment point as on one side the user's body prevents the attachment falling off to that side and on the other side a portion of the collection bag extending (in use) above the connecting piece on the collection bag.

In use of a hanging attachment such as a securing loop, the length of the loop and the receptacle body are adapted so that when the receptacle is hung in position the waste discharge outlet of the collection bag extends sufficiently into (and through) the inlet opening of the receptacle body to avoid spillage (all material goes directly into the waste receiving chamber). In any event, in all embodiments, (when discharge is taking place) it is desired that the waste discharge outlet of the collection bag extends sufficiently into the inlet opening of the receptacle body to avoid spillage. The hanging attachment can be constructed of any suitable materials such as one or more of the materials set out above. It may advantageous to include an elasticated portion in the hanging attachment. This will allow for bias of the receptacle toward the collection bag. The elasticated nature will tend to pull the receptacle toward the collection bag against any counter forces such as those experienced on emptying.

In all of these arrangements the potential spillage of materials may be avoided.

The receptacle body may be self-securing to the collection bag at a plurality of positions. For example the receptacle body may be self-securing to the collection bag at a position to the front of the collection bag and as a position to the rear of the collection bag. In one arrangement the securing means extends about the periphery of the collection bag, either entirely or at least from the front to the rear (about one side portion).

Such self-securing of the receptacle body helps to ensure that no bodily waste materials are accidentally spilled during the discharge process from the collection bag. Where opposing sidewalls (for example front and rear) are provided on the receptacle body it is desirable that the sidewalls are self-securing to opposing sides of the collection bag such as in the manner described above.

When the receptacle is secured to a collection bag , the end user may stand away from the disposal unit while emptying the collection bag. Alternatively, the end user may sit on a toilet or chair or the like to empty the collection bag. One the contents of the collection bag has been taken into the receptacle, the receptacle may be disposed of.

The present inventors envisage at least two types of receptacle which may be used conveniently by a bag wearer. In the first embodiment of the present invention the receptacle forms a container which is designed to hold the bodily waste materials received from discharge of the collection bag. In this first embodiment the base end of the disposable receptacle will be closed. The bodily waste materials and collected for subsequent disposal with the receptacle.

In a second aspect of the invention the receptacle takes the form of a discharge chute or funnel which may not hold the bodily waste materials for any length of time but discharges them directly. In this embodiment the base end of the disposable receptacle will be open to allow the discharge therefrom. The bodily waste materials are not then retained within the receptacle to any substantial extent. The receptacle may be disposed after use thereof. In one arrangement to provide weight for flushing purposes there may be provided an internal pocket arrange to catch and retain some of the bodily materials. Any such retained material will facilitate better flushing of the reservoir as compared to the reservoir alone.

In all embodiment of the present invention it is desirable that the receptacle is toilet-flushable. In this respect it is desirable that the receptacle is constructed of a material which will degrade naturally over time. Such materials as may be disposed of by means of toilet flushing, are well known to the person skilled in the art. Typical materials which may be used include degradable fibrous materials such as those based on cellulosic materials for example paper. Other biodegradable materials may also be employed such as biodegradable plastics. For example a “nappy sack” (diaper sack) type material may be employed.

It will be appreciated that the receptacle must be sufficiently resistant to degradation for a sufficient period of time in contact with the bodily waste materials so as to allow a user to use the receptacle without fear of premature disintegration thereof and potential resultant contamination from leaking bodily waste materials. Suitable materials for the receptacle include plastics films, for example plastics films with a paper backing (desirably at least the paper is water soluble). One such material is a polyvinyl (dissolve) film with a backing of non-woven water-soluble paper. A water resistant paper such as ‘grease proof’ paper may also be utilised. The materials may be from 26-32 gsm bleached, unbleached, colored greaseproof paper. Grease proof paper may be obtained for example from Fort James Ireland Ltd, Raytex Greaseproof Paper Fort James Ireland Ltd McKee Avenue Finglas Dublin 11 Phone no 185032334. Examples of commercially available materials include MonoSol AF M1030 75 μM polyvinyl alcohol film sold by MonoSol, LLC 1701 County Line Road Portage, Ind. 46368 USA (European Agent Greensol 28 Rue des Grahuches Vauguillettes 89100 Sens France) with a separate layer of non-woven water soluble backing paper (fibre); MonoSol A127 50 μM polyvinyl alcohol film sold by MonoSol, LLC 1701 County Line Road Portage, Ind. 46368 USA ( European Agent Greensol 28 Rue des Grahuches Vauguillettes 89100 Sens France) with a separate layer of non-woven water soluble backing paper (fibre) ; and Aicello Solublon PVAL sold by Syntana GmbH, Xantener Str. 1, D-45479 Mulheim/Ruhr Germany films such as BP, LA and NP grades with greaseproof paper backing which may be laminated, or with a separate layer of non-woven water soluble backing paper (fibre) for example as sold by Syntana GmbH , Mulheim/Ruhr Germany.

Generally, it is desirable that the receptacle is collapsible from an expanded state for use to receive the discharged waste materials to a collapsed state for storage, In this respect the receptacle will be constructed of a material which allows it to be moved between the collapsed and expanded states. The materials mentioned above are suitable in this respect also.

It is desirable that the collapsed state of the receptacle is a substantially flat condition thereof. This means that the receptacle can be discreetly and easily carried by a user of a collection bag. The receptacle is thus very easy to use at various locations. Users do not need to concern themselves with having specialised equipment available for emptying of the collection bag.

It is desirable that the elongate body of the receptacle becomes narrower in a direction from the top end thereof towards the base end thereof. In the embodiment where the base end of the receptacle is closed and forms a container, the narrowing of the receptacle body provides the receptacle with a flushable profile. In the embodiment where the receptacle acts as a funnel the narrowing of the receptacle body allows for more control of the discharge of the bodily waste materials through the open base end of the receptacle.

In any event it will be appreciated that the receptacle may be provided with a closure for its inlet opening. Such a closure again mitigates against spillages etc. Such a closure may be provided by a folding piece (for example a flap) on the receptacle body. The closure may be provided with securing means (of the self-securing type in particular) to hold it closed. The closure may fold across the inlet opening to provide a closure therefor.

In one arrangement, the receptacle has opposing walls which are joined by interposed collapsible wall portions so as to form the elongate body thereof. The collapsibility of the collapsible wall portions may be provided by at least one longitudinal fold line running along each of the collapsible wall portions. The wall portions which collapse by folding about the fold line is provided. Suitably, a plurality of fold lines are provided on each of the collapsible wall portions. Where a plurality of fold lines are provided it is desired that folding occurs in what is known as a fan-type, a fluted, a bellows-type or accordion-type folding arrangement. In such an arrangement the parts of the wall portions between successive fold lines collapse or stack upon each other when a compression force to the collapsed state is experienced. Desirably an odd number of fold lines are provided for example 3, 5, 7 or 9.

In an alternative embodiment, the side walls may be configured in such a way so that they do not have fold lines. For example the side walls may be formed by sealing the edges of the opposing walls (front and rear walls). Suitably the side walls may be formed by heat sealing the edges of the opposing walls.

Indeed it is desirable that a resting state of the receptacle is its collapsed state. In such a case, a user must exert a force to move the receptacle to its working configuration.

As described above, a sanitary ware item securing means such as a sanitary disposal unit or ware item securing means, for example a flap may be provided on the body the securing means being arranged for securing the receptacle to the sanitary ware item such as a toilet for example a seat or rim thereon. It will be appreciated that in embodiments having both a sanitary ware item securing means and a collection bag securing means that the collection securing means and the sanitary ware item securing means may be spaced apart along the receptacle body.

It is desirable that the sanitary ware item securing means is self-securing to the sanitary ware item. In this respect it may be provided with adhesive for self-adhering to be sanitary ware item. For example a peel-off tab or cover may be provided over an adhesive patch on the sanitary ware item securing means. Removal of the peel off tab exposes the adhesive. The sanitary ware item securing means may be provided in the form of a flap.

Generally the sanitary ware item securing means or flap will be constructed of the same or similar materials to the receptacle. In this respect it is desirable that the sanitary ware item securing means or flap has a first (storage) position where it is substantially flush with the receptacle body and a second (working) position where it projects outwardly from the receptacle body for securing to a sanitary ware item.

The sanitary ware item securing means or flap may be integrally formed with the receptacle body or may be constructed as a separate piece which is attached thereto. Desirably the sanitary ware item securing means is a flap which is a flat piece which folds about a fold line substantially transverse to the receptacle body. It will have generally a first end attached to the receptacle body and a second free end. Desirably the fold line is along the junction between the (first end of the) sanitary ware item securing means or flap and the receptacle body so that the sanitary ware item securing means or flap can lie flat against the receptacle body (and thus substantially flush therewith) or be moved to project at an angle away therefrom. In the working position of the sanitary ware item securing means or flap it will generally project (radially outwardly) to one side of the receptacle body. The sanitary ware item securing means or flap will desirably be provided on one of the opposing walls of the receptacle.

Generally, it will be an underside of the sanitary ware item securing means or flap which is secured to the sanitary ware item.

Desirably, when the receptacle is secured to a sanitary ware item means by a securing means the end user may sit on the sanitary ware item item such as a toilet and empty a collection bag.

For the further convenience of the user of the receptacle, it is desirable to provide at least one finger grip portion which projects away from the receptacle body about the inlet opening. For example one or more gripping tabs may be provided on the receptacle body. Suitably, any such finger grip portion or gripping tab that is provided at the mouth of the receptacle about the inlet opening. This is convenient in particular where the receptacle is collapsible. In such an instance the finger grip portion or tab allows for ease of movement of the container into its working state. Where a plurality of finger grip portion or tabs are provided it is desirable that at least two diametrically opposed finger grip portion or tabs are utilised (for example to pull apart front and rear walls) This facilitates gripping of opposing sides of the receptacle by respective hands.

It will be appreciated that the receptacle is of a length to extend between the collection bag and into a disposal unit such as a toilet, for example to a space below any toilet seat and in any event desirably below the rim. In particular sufficient length is allowed so that the possibility of waste discharged (particularly pertinent for the funnel arrangement) missing the toilet bowl due to movement of the receptacle during discharge.

In one arrangement a guard portion is provided on the receptacle which is attached to the receptacle adjacent the inlet opening and which is arranged to project from the receptacle to a position alongside the collection bag. In some embodiments the connection portion may include the securing means described above. In this way the guard portion may act as a splash guard. The guard portion may (in addition to, or in the alternative to, the collection securing means mentioned above) be provided with securing means to attach the guard portion to the collection bag. Adhesive securing means are particularly desirable in this context.

In one arrangement a connection portion is provided on the receptacle for connection to the collection bag. The connection portion desirably takes the form of a wrap around which is for wrapping about and connecting to the collection bag. For example if the collection bag has a neck portion with a discharge outlet the connection portion may be adapted for fitting to (and desirably about)that neck portion. It will be appreciated that the splash guard may form the or part of the connection portion. It is desirable that the splash guard and/or the connection portion are provided with securing means. In an alternative embodiment, the connection portion and receptacle may be separate independent parts. In such an embodiment the connection portion may be provided with two securing areas; one to secure the connection portion to the receptacle and one to secure the connection portion (or the combined connection portion-receptacle) to the collection bag. When the connection portion has been secured to the receptacle, the connection portion-receptacle arrangement may function in the same manner as the embodiments previously described above.

It will be appreciated that any one or more of the above features may be provided in combination.

The invention extends to a receptacle substantially as described herein with reference to the drawings.

### BRIEF DESCRIPTION OF THE DRAWINGS

The present invention will be more clearly understood with reference to the accompanying drawings in which:

FIG. 1A is perspective view of a disposable bag part of a receptacle of the present invention;

FIG. 1B is a perspective view of another disposable bag part of a receptacle of the present invention;

FIG. 1C is a perspective view of a different disposable bag part of a receptacle of the present invention;

FIG. 1D is a perspective view of an alternative embodiment of a disposable bag part of a receptacle of the present invention;

FIG. 1E is a perspective view of an alternative embodiment of a disposable bag part of a receptacle of the present invention;

FIG. 2A is a perspective view of a disposable receptacle and securing means of the present invention;

FIG. 2B is a perspective view of an alternative embodiment of a disposable receptacle and securing means of the present invention;

FIG. 2C is a perspective view of a further alternative embodiment of a disposable receptacle and securing means of the present invention;

FIG. 2D is a perspective view of an alternative embodiment of a disposable receptacle and securing means of the present invention;

FIG. 2E is a perspective view of an alternative embodiment of a disposable receptacle and securing means of the present invention;

FIG. 2F is a perspective view of a connection portion of the present invention particularly suitable for use with the bag part of a disposable receptacle of FIGS. 1A to 1E;

FIG. 2G is a perspective view of a waste collection bag being positioned and secured in one embodiment of the disposable receptacle of the present invention;

FIG. 3 is an illustration of one of the embodiments of the disposable bag part of a receptacle of the present invention being secured on a sanitary ware item seat.

The drawings will now be described in detail.

### DETAILED DESCRIPTION OF THE DRAWINGS

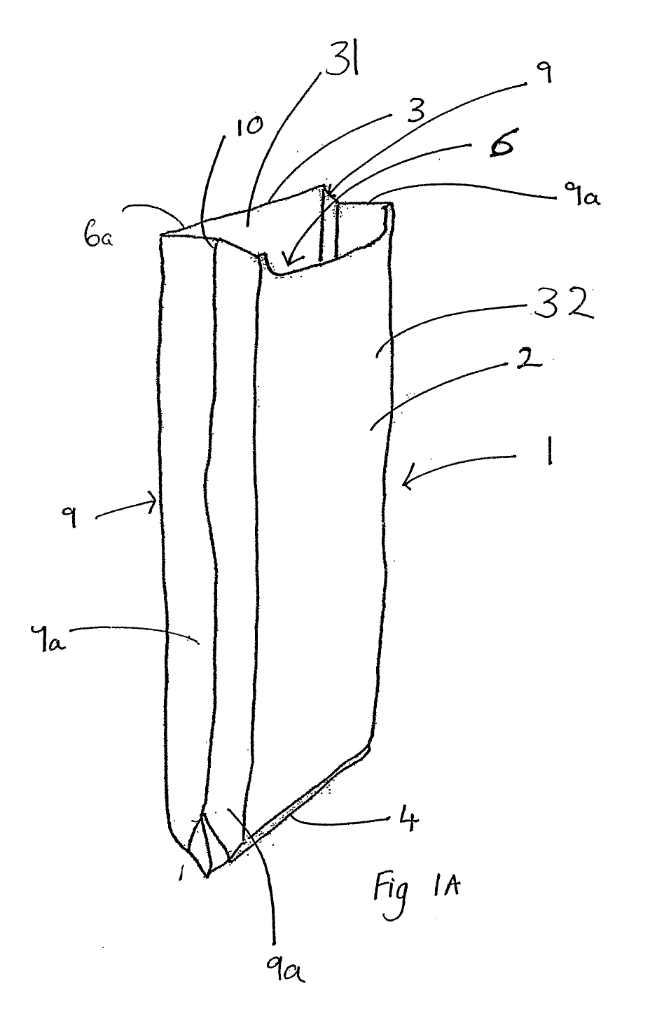

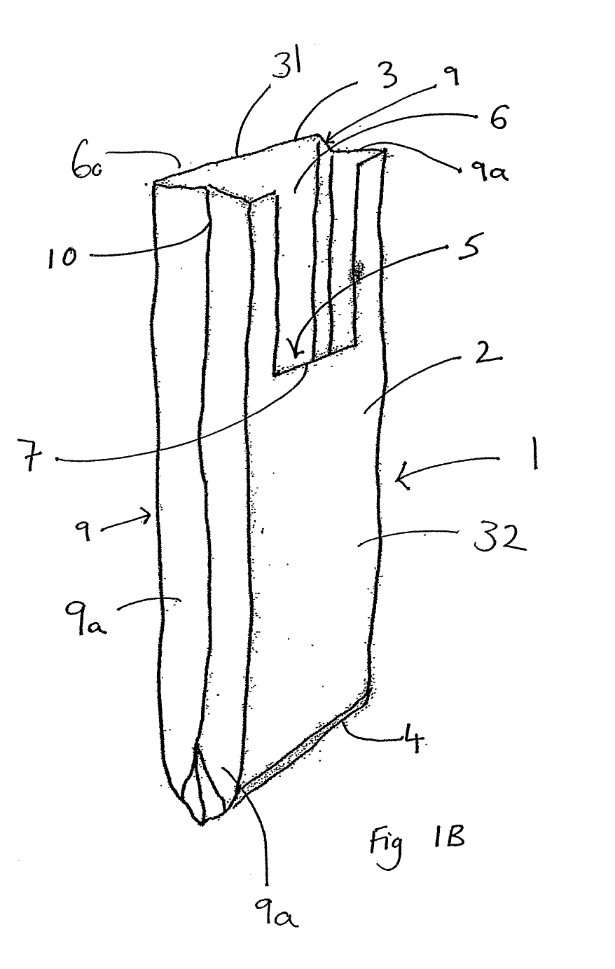

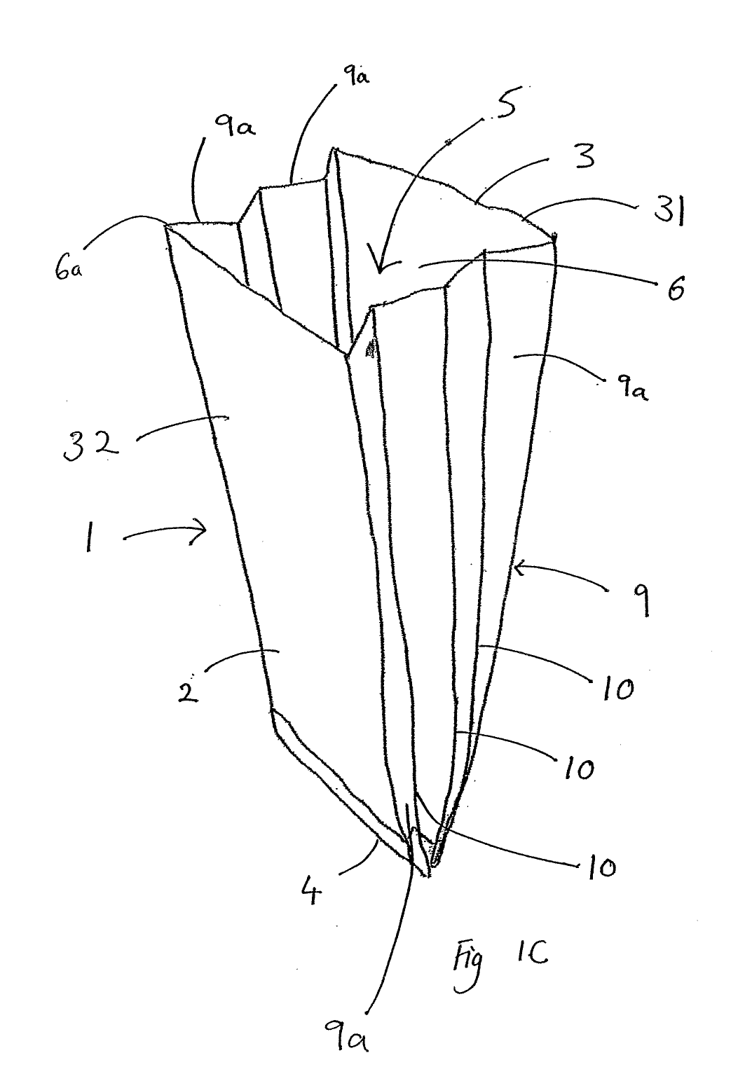

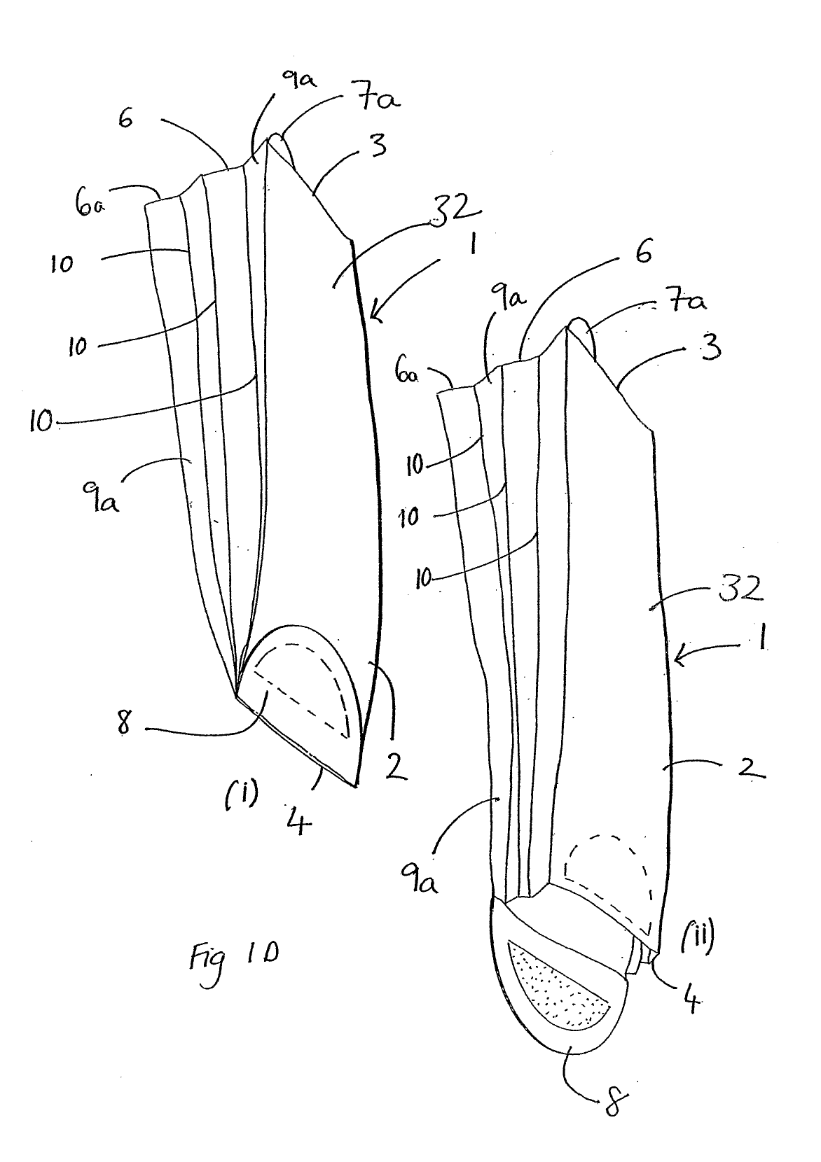

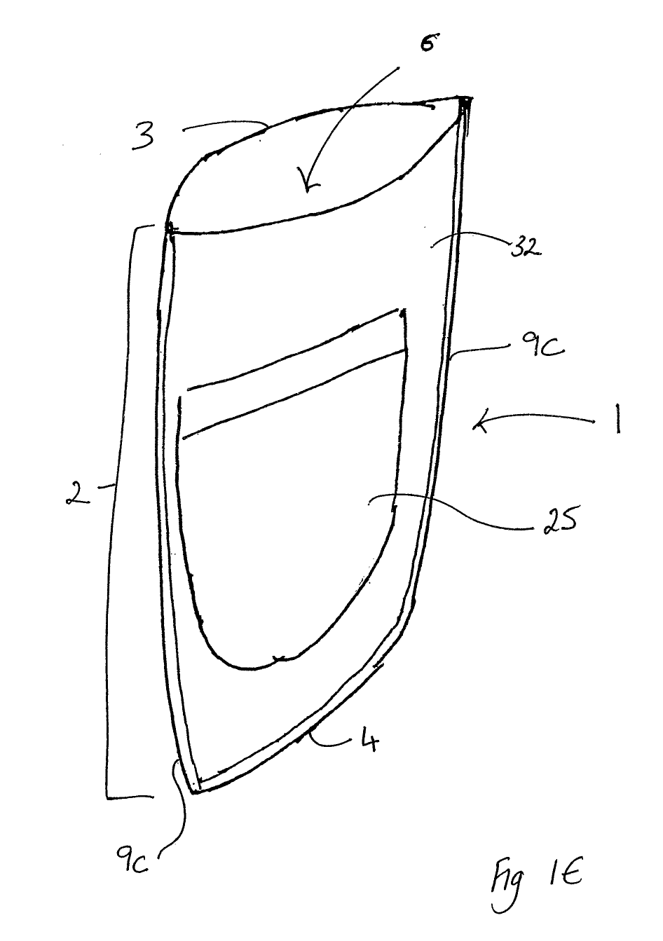

The bag part 1, of a disposable receptacle of the present invention, comprises a number of alternative embodiments, see FIGS. 1A to 1E. All of these Figures show bag parts as the securing means to attach them to a collection bag may vary. Any bag part may be employed with any securing means or suitable securing means are described below. All of the bag part 1 of the disposable receptacles have key features in common including an elongate body 2. The elongate body 2 has a top end 3 and a base end 4. The bag 1 is formed with an internal waste material-receiving chamber 5 formed between the top end 3 and the base end 4. The elongate body 2 and waste material-receiving chamber 5 are of suitable dimensions so that they can receive waste material from a bodily waste discharge collection bag (such as a colostomy or an ostomy collection bag) without overflowing. For example, the internal dimensions of the waste material-receiving chamber 5 are adequate to receive the volume of waste material discharged from the collection bag. The bag part 1 of the disposable receptacle of the present invention can be manufactured in a number of sizes. For example smaller sized disposable bags 1 may be suitable for use by children or persons with a requirement for small discharge collection bags. Whereas an adult user may require a larger bag part 1 to a disposable receptacle as the volume of waste that their discharge collection bag holds will be greater. Typical dimensions of the disposable receptacle are in the region of 36 cm×16 cm to 20 cm×6 cm, such as 30 cm×12 cm to 24 cm×8 cm, for example 28 cm×10 cm to 26 cm×10 cm. Disposable receptacles of the present invention may be marked, for example colour coded to indicate the maximum volume the waste material-receiving chamber 5 can accommodate.

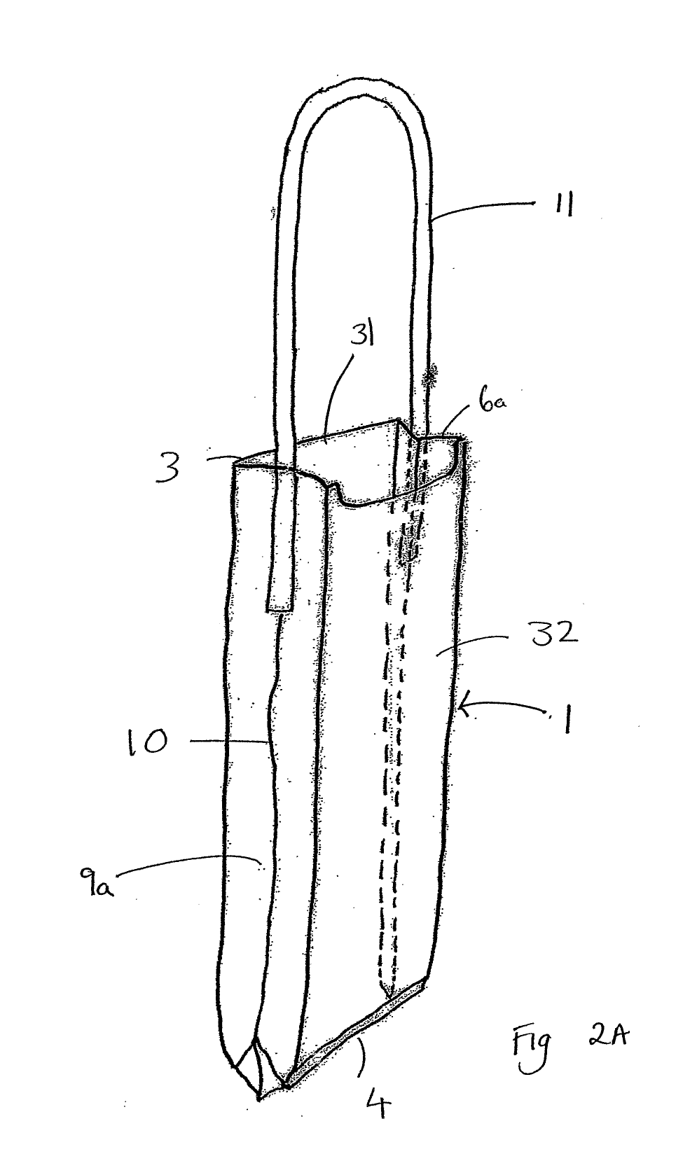

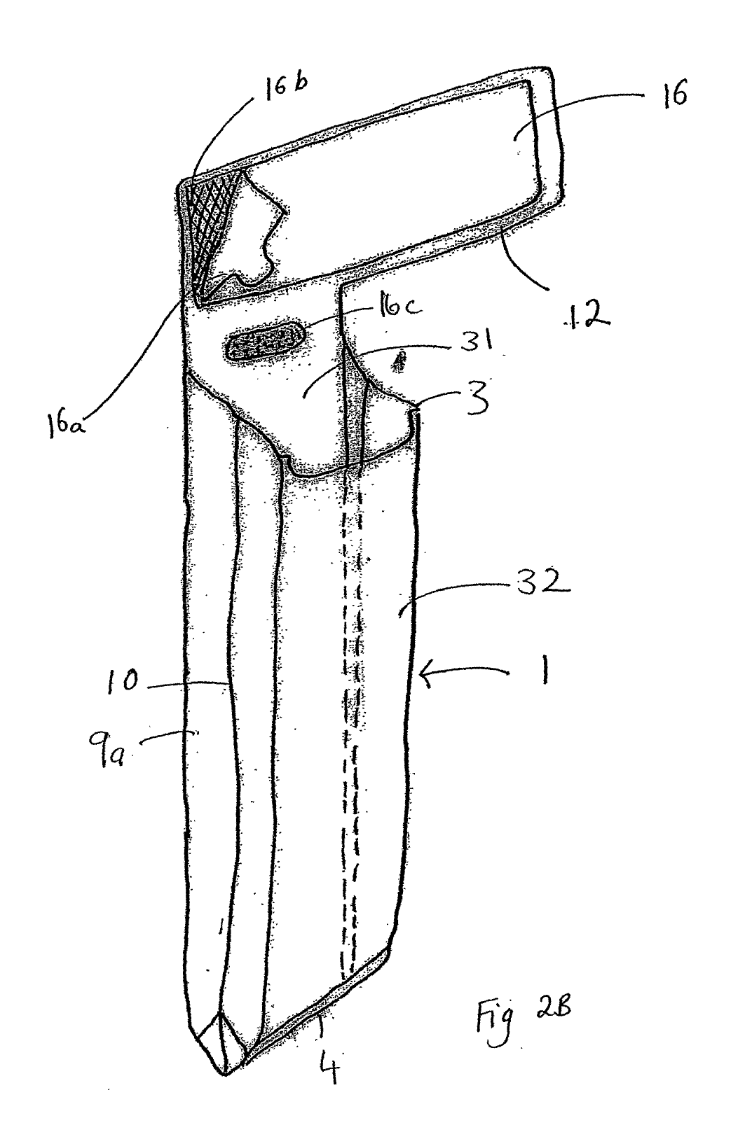

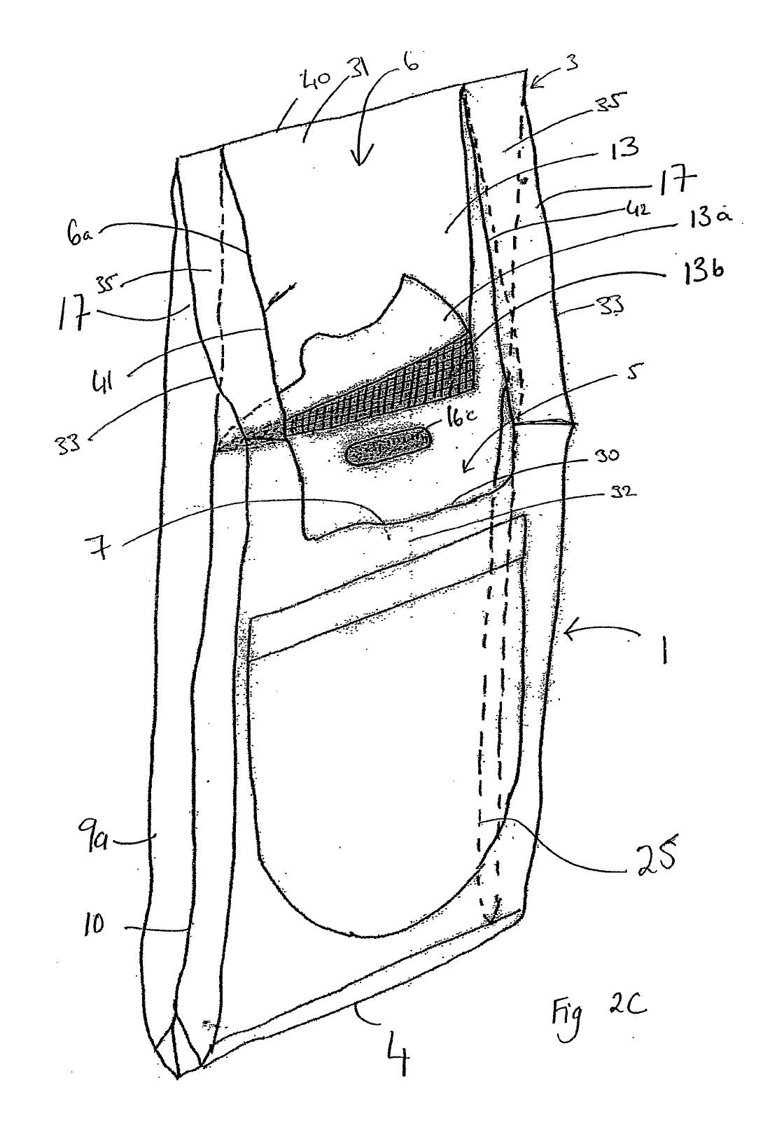

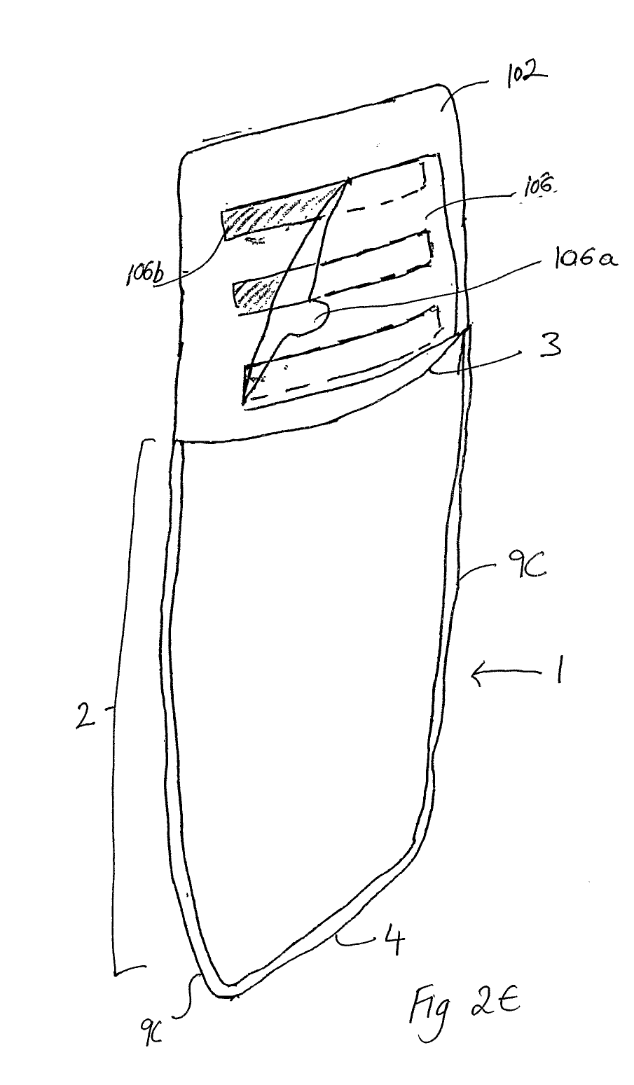

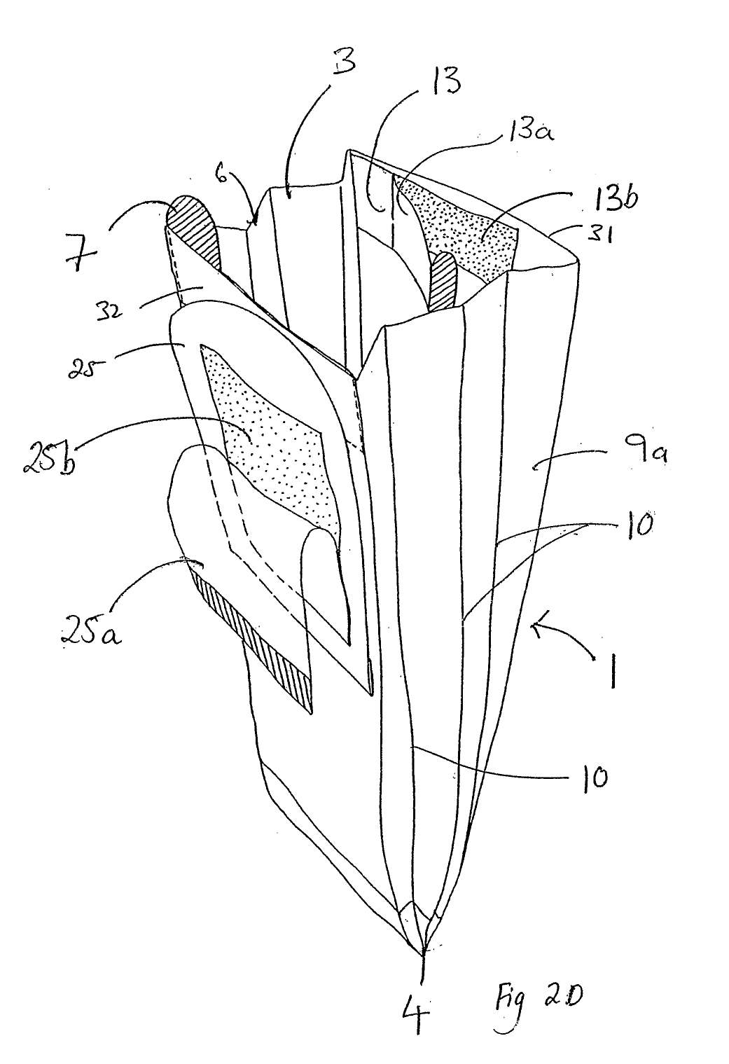

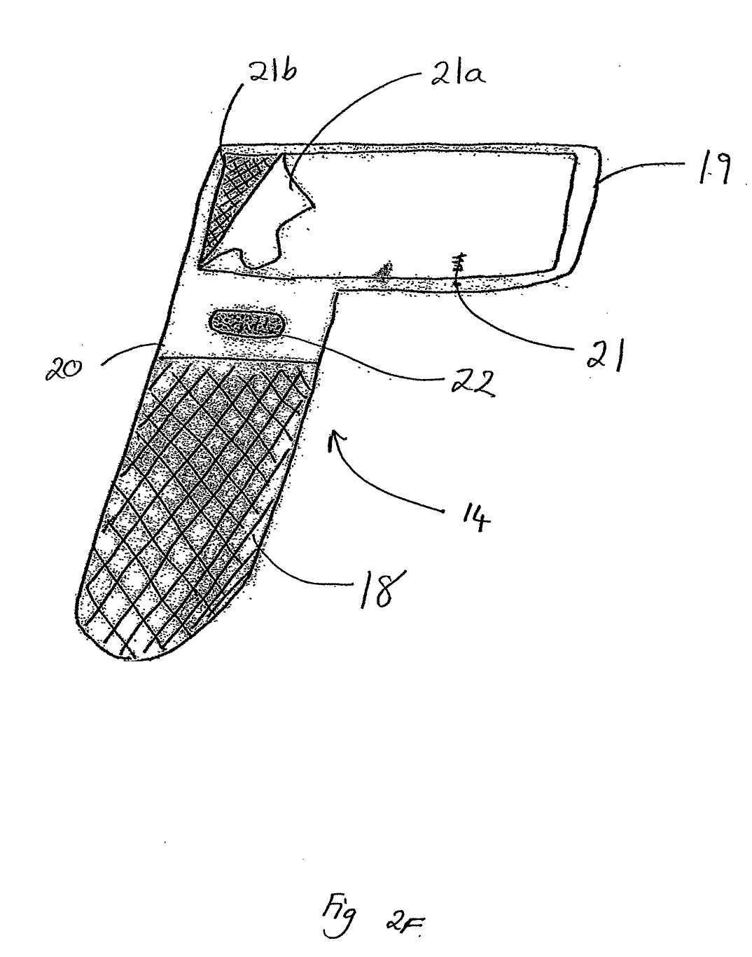

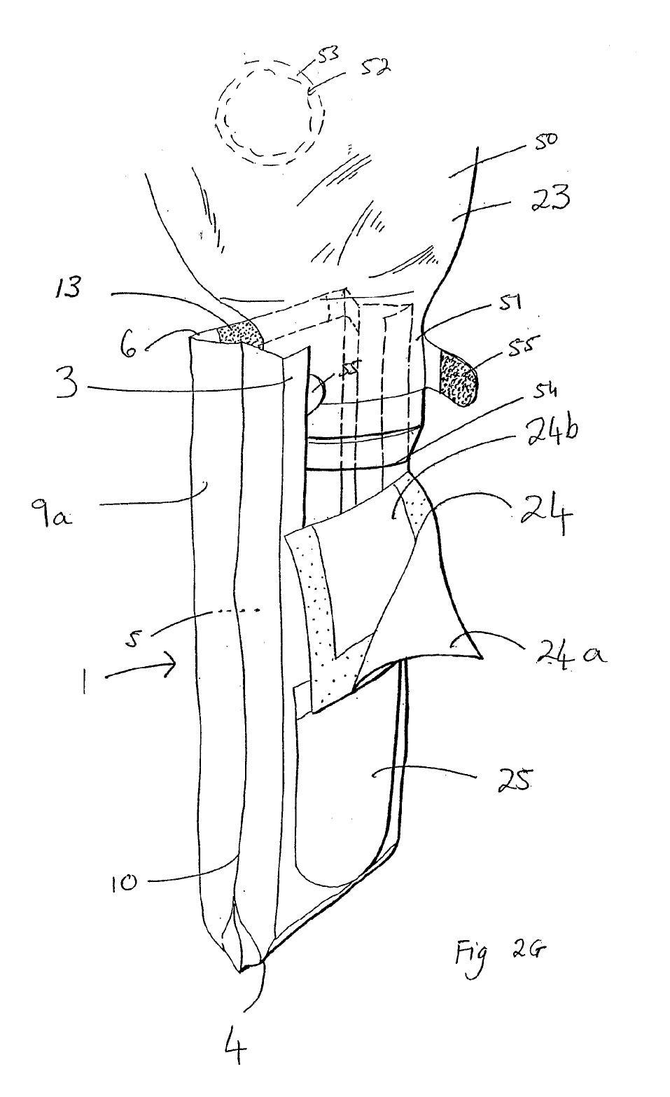

The bag part 1 of the disposable receptacle has an inlet opening 6. The inlet opening 6 is formed by a mouth 6a about the top end 3 of the elongate body 2. In the embodiments the inlet opening 6 is at the top end 3 of the elongate body 2 but of course could alternatively be located proximate thereto. The inlet opening 6 is adapted to receive the outlet of the collection bag in a manner analogous to that shown in FIG. 2G. Preferably, the inlet opening 6 is configured so that the outlet of the collection bag can sufficiently extend into the waste material-receiving chamber 5. In such a configuration, the risk of spillage of the waste material as the collection bag is emptied is minimised,

In some embodiments of the bag part 1 of the disposable receptacle, at least one finger grip portion 7 is provided (FIGS. 1B and 1D). The at least one finger grip portion 7 is employed for end user convenience. In the embodiment of FIG. 1B the finger grip portion takes the form of a recess. In the embodiment of FIG. 1D the finger grip portion 7 takes the from of a tab which is desirably positioned so that it projects away from the opening. In one embodiment, such as that in FIG. 1B, the finger grip portion 7 is formed by a wall adjacent a recess or aperture in the body 2 which is positioned proximate to the inlet opening 6 and is contiguous therewith. In FIG. 1B, the finger grip portion 7 is provided by the mouth 6a of the inlet opening 6. In the embodiment illustrated in FIG. 1D, the finger grip portion 7 is formed by a tab 7a that is connected to the top end 3 of the body 2. In an arrangement where the opening 6 is not at the top end of the body 2 the finger gripping portion 7 may be positioned proximate to the inlet opening 6. The finger gripping portion 7 assists the end user in opening the mouth of the disposable receptacle for reception of waste. Disposable receptacles of the present invention may have finger gripping portions 7 such as large tabs 7a to aid end users with a lack of dexterity (such as the elderly, patients with inflammation or chronic conditions such as arthritis, or the disabled) in opening the receptacle. Disposable receptacles of the present invention may have an indication such as a colour coding or the like to indicate the intended end user size etc.

It is desirable that in some embodiments, the finger gripping portion 7 is distinguishable from the rest of the body 2 to assist end users who have poor eye sight to locate it. For example, the finger gripping portion 7 may be a different colour to the rest of the body 2. Additionally or alternatively the finger gripping portion 7 may be a different texture to the rest of the body 2.

The base end 4 of the receptacle may be formed with a blind or closed end as in the embodiments in FIGS. 1A, 1B, 1C and 1E. In those embodiments there is no opening in the lower end of the receptacle. The receptacle usually then completely retains the waste material. In such an arrangement, the receptacle provides a chamber which has a bag like structure into which the waste material from the collection bag can be emptied. The disposable receptacle is preferably disposable by flushing it down a toilet. The weight of the waste material in the receiving chamber 5 will assist in the flushing of the receptacle. The receptacle may also be disposed of by other means such as by placing in another container for example a “nappy sack”. The lower end such as the base end 4 of the receptacle has a tapered shape. The tapered shape lends a flushable profile to the receptacle.

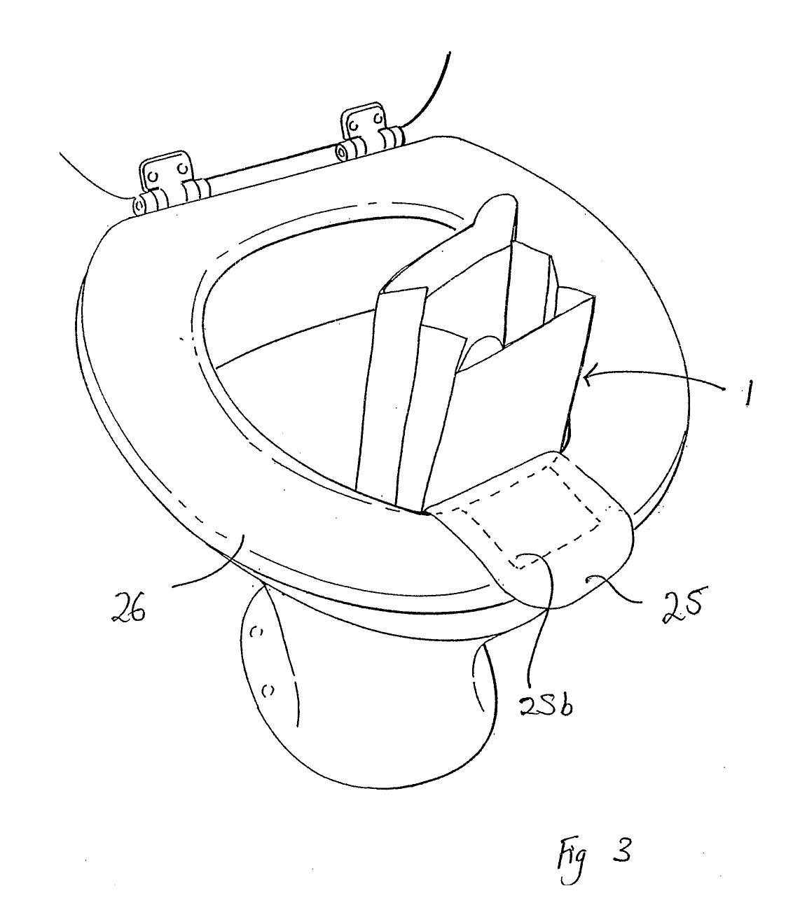

In an alternative embodiment illustrated in FIG. 1D, the receptacle may have an operable (and sealable) flap 8 at its base end 4. The sealable flap 8 may be held closed by self securing arrangements such as (tacky) adhesive or Velcro™ or the like. The sealable flap 8 may be opened and closed more than once. The end user can then decide whether they wish to use the receptacle as a bag and maintain the flap 8 in the sealed position (FIG. 1D(i)). Alternatively, the end user may desire to utilise the receptacle as a funnel or chute (FIG. 1D(ii)). In the funnel or chute arrangement the base end 4 is not sealed. The body 2 of the receptacle is of a sufficient length to extend from the outlet of the collection bag into the toilet bowl (see FIG. 3). Desirably, to discharge the base end 4 of the receptacle is located below the rim of the toilet bowl. Waste material is discharged from the collection bag directly into the toilet bowl. A user may be seated and still utilise the receptacle of the invention.

The length of the receptacle will vary depending on the size of the end user. For example a child would require a smaller length compared to an adult. Typically, the length of the receptacle would be in the region of 34 cm to 20 cm, such as 32 cm to 24 cm, for example 30 cm to 26 cm. The product may be colour coded to indicate its length. Once the waste material has been discharged into the toilet bowl, the receptacle can be disposed off. The receptacle may be disposed of by flushing it down the toilet. Alternatively, the receptacle may be disposed of by a different method such as being placed in a bin optionally within another container. In one embodiment, an internal pocket may be provided in the waste material-receiving chamber 5. This is particularly advantageous when the receptacle is to be used in a funnel arrangement, as some of the waste material may be retained in the internal pocket to provide weight to the receptacle for flushing purposes.

The desired way of disposing of a used receptacle is by flushing it down a toilet. The receptacle is preferably made of any suitable biodegradable material which can be disposed of by flushing in an environmentally friendly way. Desirably the receptacle is made of a cellulosic type material. The material of which the receptacle is constructed must have sufficient resilience so that it does not disintegrate when the waste material enters the receptacle. Suitable materials for manufacturing the receptacle include plastics films, for example plastics films with a paper backing (desirably at least the paper is water soluble) . One such material is polyvinyl dissolve film with a backing of non-woven water soluble paper. A water resistant paper such as ‘greaseproof’ paper may also be utilised. The materials may be from 26-32 gsm bleached, unbleached, colored greaseproof paper. Grease proof paper may be obtained for example from Fort James Ireland ltd, Raytex Greaseproof Paper Fort James Ireland Ltd McKee Avenue Finglas Dublin 11 Phone no 185032334. Examples of commercially available materials include MonoSol AF M1030 75 μM polyvinyl alcohol film sold by MonoSol, LLC 1701 County Line Road Portage, Ind. 46368 USA ( European Agent Greensol 28 Rue des Grahuches Vauguillettes 89100 Sens France) with a separate layer of non-woven water soluble backing paper (fibre); MonoSol A127 50 μM polyvinyl alcohol film sold by MonoSol, LLC 1701 County Line Road Portage, Ind. 46368 USA ( European Agent Greensol 28 Rue des Grahuches Vauguillettes 89100 Sens France) with a separate layer of non-woven water soluble backing paper (fibre); and Aicello Solublon PVAL sold by Syntana GmbH , Xantener Str. 1, D-45479 Mulheim/Ruhr Germany films such as BP, LA and NP grades greaseproof paper backing which may be laminated or with a separate layer of non-woven water soluble backing paper (fibre) for example as sold by Syntana GmbH Xantener Str. 1, D-45479 Mulheim/Ruhr Germany.

The receptacle may be an immediately disposable device as described above or may be utilised a small number of times. The receptacle may also be a portable device. The receptacle preferably has two states; a collapsible state and an expanded state. The collapsible state may be used for storage. The expanded state (shown in all Figures except for FIG. 2F) is used for emptying waste material from the collection bag. Desirably, the receptacle is constructed from suitable material that allows it to be moved between the collapsed and extended states. Suitable materials includes those listed above. Desirably the receptacle has opposing walls 9 such as front 32 and rear 31 walls. In some embodiments the opposing walls 9 are of different heights. The opposing walls 9 may be joined by interposed collapsible wall portions 9a. The collapsibility of the wall portions 9a may be provided by longitudinal fold lines 10 running along each of the collapsible wall portions 9a. In the embodiment shown in FIGS. 1A, 1B, 2A, 2B and 2C the collapsible wall portions 9a have one longitudinal fold line 10. Whereas the embodiment illustrated in FIGS. 1C, 1D, 2E and 3 have three longitudinal fold lines 10. The number of fold lines 10 in the collapsible wall portions 9a will generally be an odd number. Desirably, collapsible wall portions 9a with a plurality of longitudinal fold lines 10 such as those illustrated in FIGS. 1C, 1D, 2E and 3 open in a fan shape or concertina shape arrangement. By providing a plurality of collapsible wall portions 9a and a plurality of longitudinal fold lines 10 one can easily create a receptacle which is wider at its top end 3 as compared with the top end 3 opening of a receptacle with only one longitudinal fold line 10. Receptacles 1 with a wider opening at the top end 3 may be adapted for use by people with little dexterity. A wider opening at the top end 3 will allow greater access to the interior of the receptacle and this allows for ease of use for disposal of materials from a collection bag.

An alternative embodiment is illustrated in FIGS. 1E and 2E in which the wall portions 9C are formed by sealing the edges of the front 32 and rear 31 walls. The edges of walls 31 and 32 may be sealed by any suitable means. One suitable sealing means is heat sealing.

FIGS. 2A to 2G illustrate a plurality of embodiments of suitable securing means 11, 12, 13, 14, 16c, 22, 102 of the present invention which together with the bag portions of FIGS. 1A to 1E may form the receptacle of the invention in any desired combination. Any suitable securing means may be employed, and the securing means suitable for use with the present invention may be varied and include all suitable fasteners, adhesives and the like. Any suitable size or shape may be used. The securing means 11, 12, 13, 14, 102 are adapted to secure the receptacle to the collection bag and additionally comprise connection portions as set out below. Preferably the securing means 11, 12, 13, 14, 102 are detachably attachable to the collection bag as it may be required to empty the collection bag several times a day using one or more of disposable receptacles of the present invention. The respective securing means 11, 12, 13, 14, 102 function as a safety feature of the present invention to minimise the likelihood that the discharge chute of the collection bag will be displaced from the waste-material receiving chamber 5 during emptying of the collection bag. As can be seen, the securing devices 11, 12, 13, 14, 102 are adapted to be easy to use by the end user.

The securing device illustrated in FIG. 2A consists of a hanging attachment or loop 11. The loop 11 is provided to hold the receptacle in position relative to the collection bag. Loop 11 is arranged to hang from any suitable location such as from the collection bag. In use, the loop 11 may be moved over the top of the collection bag so that the loop 11 is supported by the attachment of the collection bag to the patient's body (usually a mated connection between the collection bag and the mount on the upper body). The loop 11 is constructed from the same material as the body 2. Alternatively, the loop 11 may be constructed from an elasticated material. In use, the loop 11 is hung from a suitable support and with a discharge outlet of the collection bag over or within the mouth of the container. The end user opens the discharge outlet of the collection bag so that the waste material is expelled from the collection bag into the receptacle (see FIG. 2F for an illustration of the emptying process). Once the collection bag has been emptied, the end user can re-seal the collection bag (generally collection bags are reusable/resealable) and dispose of the receptacle.

The securing means 12 illustrated in FIG. 2B is a self securing means. In the embodiment, the securing means 12 takes the form of an elongate flap or strip connected to (and projecting from) the top end 3 of the receptacle. The strip 12 houses a self securing means 16. The securing means 16 comprises a connection portion formed by the flap and a covered adhesive patch (peel-away covered) thereon. The self securing means 16 may be a peel off adhesive strip such as in the embodiment shown where a discardable peel off layer 16a is present. In such an embodiment, when the discardable layer 16a is peeled off, it reveals an underlying adhesive patch 16b. The adhesive patch 16b may be used to secure the top end 3 of the receptacle to the collection bag. In particular, in the embodiment illustrated, the strip 12 is elongate so that the strip 12 (will extend about) may be wrapped about the collection bag to provide extra grip (security). The securing means 16b is desirably a (tacky) low strength adhesive. The adhesive 16a is strong enough to secure the receptacle to the collection bag whilst allowing the end user to easily remove the adhesive strip 12 from the collection bag once the collection bag has been emptied. Suitable adhesives for use with any embodiment of the invention include: dispersal adhesives such those available from Henkel™ under the product name Adhesin J1125, J1610 or J1620; Hot melt adhesives such as those available from Henkel™ under the product name Sanicare HM6410; and double sided adhesive tapes such as those available from 3M™ under product identifiers 9415, 9416 and 9425; and PPI type 228 available from PPI Adhesive Products Ltd Waterford Industrial Estate Cork Road Waterford.

An additional securing means in the form of a fastener 16c is provided. The fastener 16c, which in the embodiment is of the hook and loop fastener type, is adapted to (and arranged to) engage with a corresponding fastener on the collection bag. Such a fastener is typically provided on the collection bag to hold a discharge spout thereon in a folded up or retracted/closed position. Neither securing means interferes with the discharge of materials from the collection bag to the receptacle. In this embodiment, and or is desirable, the securing means is arranged to retain the collection bag in a discharge configuration thereof.

The receptacle of the invention shown in FIG. 2C has a self securing patch 13. In the configuration shown the top end of the container 3 is adapted for ease of emptying of the collection bag. In particular opposing walls of the receptacle are of different heights. This means that the mouth 6C of the inlet opening 6 has a lower edge 30, an upper edge 40, which are joined by opposing (side) edges 41, 42. In this way a guard portion is formed. In the embodiment the rear wall 31 of the receptacle is taller than the front wall 32. This type of arrangement where one of two opposing walls is taller than the other is useful because a substantial portion of the taller wall 6 can be adapted to extend past the discharge outlet of the collection bag (to allow securing to the collection bag) while the lower wall allows the user to discharge materials without interference as the user can easily access the collection bag from the lower side. The securing means 16c is on the guard portion A region of the front wall 32 may provide a finger grip portion 7 to assist the end user in opening the receptacle from a collapsed to expanded state. In the embodiment illustrated in FIG. 2C, the securing means comprises a disposable peel off cover 13a upon an adhesive patch 13b. In use the end user may peel off the cover 13a to reveal the adhesive 13b. When the adhesive 13b is exposed it provides an area that can be used to secure the receptacle to the collection bag.

As described above the embodiment of FIG. 2C has walls configured to minimise the risk of splash back or spillage when the collection bag is being emptied. Side walls 17 may be of any suitable shape and dimensions but as is shown it is desirable that a top end 33 runs from the front to the rear wall. Desirably, the side walls 17 can be considered wedge shaped. As with other embodiments, the side walls 17 comprise collapsible wall portions 9a to allow the receptacle to move from a collapsed to expanded state and vice-versa. In the embodiment shown two side rims 35 are shown which run from the (top of the) side walls 17 inwardly about the mouth 6. The rims 35 also provide protection against splash-back. As with other embodiments the receptacle 1 is provided with a flap 25 which can be utilised to leave the receptacle to a toilet, such as a seat or rim thereof. Further provided on the receptacle is a further securing means 16c, which, as described above for FIG. 2B can be employed to further secure the receptacle to a collection bag. Once the collection bag has been emptied, the receptacle may be removed from the collection bag and disposed of.

FIG. 2D shows an alternative embodiment of a receptacle of the invention. The receptacle has a plurality of longitudinal fold lines 10 between collapsible wall portions 9a. As described above, the receptacle having a number of fold lines 10, about the top end 3 of the receptacle will generally be wider (this providing a wider opening) than a receptacle with only one fold line 10. In the embodiment of FIG. 2D, the finger gripping portion 7 is a (small) tab 7 protruding from the receptacle body 2. As the fan-like or concertina opening may provide sufficient access to the securing region 13 to allow a user to secure the region 13 to a collection bag, the embodiment does not show a cut out portion finger grip 7 (unlike the embodiment illustrated in FIG. 2C).

As with other embodiments a flap 25 is provided for securing the receptacle to a toilet or the like. The flap has an adhesive patch 25b covered by a peel off cover, for adhering the flap to the toilet. Similar to other embodiments the securing means 13 is on an inside surface of one of the front and rear walls (in this case the rear wall 31). It is desirable therefore to have the tab 7 arranged to move the front wall 32, in particular to allow a discharge outlet of a collection bag to be fitted into the mouth of the container. Again as with other embodiments the receptacle has a flushable profile, for example having a narrow end (base end).

The securing means 102 illustrated as part of the embodiment of FIG. 2E is a self securing means. In the embodiment, the securing means 12 takes the form of a flap or strip connected to (and projecting from) the top end 3 of the receptacle. The strip 102 houses a self securing portion 106. The securing portion 106 comprises a connection portion formed by the flap and a covered adhesive patch (peel-away covered) thereon. The self securing means 106 may be a peel off adhesive strip such as in the embodiment shown where a discardable peel off layer 106a is present. In such an embodiment, when the discardable layer 106a is peeled off, it reveals an underlying adhesive patch 106b. The adhesive patch 106b may be used to secure the top end 3 of the receptacle to the collection bag.

FIG. 2F illustrates a connection piece 14 in accordance with the present invention. The connection piece comprises the securing means for securing the receptacle to the collection bag. Such a connection piece may be integrally formed with the bag to form the receptacle. In the embodiment shown it is a separate piece attachable to a bag. For example the connection portion may be re-usable while the bag is disposed of. The connection piece 14 may be ‘L’ shaped, alternatively the connection piece may be ‘I’ shaped or T′ shaped. In the ‘L’ shape configuration one limb 20 has a securing region 18. The securing region 18 may comprise any known securing means including self securing means such as Velcro™, adhesive or the like. In the embodiment an adhesive patch forms the securing region 18h. Typically, the adhesive means will have a disposable peel off cover (not shown) that when removed will reveal the adhesive patch 18. The securing means of the connection piece 14 is suitable for attaching the connection piece 14 to any embodiment of the receptacle of the present invention and/or to a bag/collection device of the type illustrated in FIGS. 1A to 1D. Another limb of the connection portion 19 comprises a further securing means 21. The securing means 21 may comprise any known securing means including self securing means such as Velcro™, adhesive or the like. The securing means illustrated comprises a disposable peel off strip 21a over an adhesive patch 21b. The securing means is adapted to secure a receptacle to a collection bag. The limb 19 is of suitable dimensions so that it can attach to the back of the collection bag (the side facing the patient's body) and at least partially wrap around the front of the collection bag (the side facing outwards from the patient's body). The connection portion 14 also has a further securing means 22 that is adapted to cooperate with the collection bag, for example attachments on certain makes of collection bags. For example the securing means 22 may be Velcro™. It is desirably that the securing means 22 inter-engage with the securing means on the collection bag. If the collection bag has a strip of Velcro™ with male connections, it is desirable that the securing means 22 is a strip of Velcro™ with female connections and vice-versa again the position of the additional securing means.

FIG. 2G illustrates how generally a collection bag of a user may be arranged for discharge into a receptacle of the present invention. The collection bag 23 is of the type comprising a flexible bag body 50, with a neck or spout position 51 (which is arranged at a lower end thereof, and an inlet 52 which has a rim or connection portion 53 adapted to receive and engage with an outlet on a patient's body. The outlet will be provided by a connecting piece, usually of plastic material which is arranged about an artificial excretory opening on the user's body. Thus the collection bag will attached to a patient's body when being discharged. However for ease of illustration, and best view, the user is not shown. The neck 51 has a discharge outlet 54 at a lower end thereon. The discharge outlet 54 is closed by rolling the neck or spout up upon itself and closing the fasteners 55 to hold the neck closed. As can be seen from FIG. 2F, the collection bag, or at least part of the neck 57 thereof, is arranged within the mouth 6 of the receptacle 1. The discharge outlet is a safe distance within the mouth (toward the chamber 5) so spillage is not likely. The receptacle has a securing means 13 (of the type previously described) for attaching to the back of the collection bag 23. The embodiment illustrated also has an access flap 24, which, in the open configuration shown in FIG. 2F allows access to the collection bag, and in particular the neck and discharge outlet thereof. In this position an end user can access the collection bag easily without hinderance. When desired the flap 24 can be closed and secured in place by folding it back (upwards) against the top end of the receptacle and the collection bag. An additional securing area on the flap is arranged to secure to the front of the collection bag 23. Preferably, the access flap has securing means which comprises a disposable peel off strip 24a that when removed reveals an adhesive area 24b.

The access flap is particularly useful as the collapsible wall portion 9a, it is desirable that the securing area 24 folds back towards the base end 4 of the receptacle. Such a configuration may provide a finger grip for the end user and may also provide easier access to the interior of the receptacle as described above.

It is desirable that for all embodiments of the present invention, the discharge outlet of the collection bag extends sufficiently into the interior of the receptacle. In this respect the receptacle may be provided with a collection chute, of the type having walls of different heights, or a chute formed by a connection portion. Sufficient placement of the discharge outlet into the interior of the receptacle may prevent unwanted spillage or splash back of the waste material.

In addition to the embodiments described above, the receptacle may comprise a sealing means suitable for sealing the top end 3 of the receptacle prior to disposal. Such sealing means may be provided by the securing means on the receptacle.

Any receptacle of the present invention may further comprise an additional securing area which may be used to secure the receptacle to a toilet (seat) 26 (see for example FIG. 3). The additional securing area 25 is desirably a self-securing means. The securing area 25 may be a flap or a tab or the like. The securing area 25 may be adapted so that it has a stored (collapsed) and an extended state. Preferably, the flap 25 has a disposable peel off strip 25a(FIG. 2D) that reveals an adhesive surface 25b for securing to the toilet seat 26. The flap 25 may be particularly useful for end users with limited mobility and/or a lack of dexterity. The flap 25 will hold the receptacle in position while the end user secures the receptacle to the collection bag.

The words “comprises/comprising” and the words having/including” when used herein with reference to the present invention are used to specify the presence of stated features, integers, steps or components but does not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

1. A disposable receptacle for receiving bodily waste materials from a bodily waste discharge outlet of a collection bag arranged to collect and retain bodily waste materials from a user's body, the receptacle comprising:
(i) an elongate body with a top end, a base end, an intermediate portion between the top end and the base end, and formed with an internal, bodily waste material-receiving chamber;
(ii) an inlet opening formed on the body, proximate to, or at, the top end thereof, the body being adapted to receive within the inlet opening the bodily waste discharge outlet of the collection bag so as to allow bodily waste materials within the collection bag to be discharged into the bodily waste material-receiving chamber; and
(iii) securing means for holding the bodily waste material-receiving chamber in position for discharge of the bodily waste materials into the bodily waste material-receiving chamber from the collection bag.

2. A receptacle according to claim 1, wherein the securing means is for securing the material-receiving chamnber relative to the collection bag.

3. (canceled)

4. (canceled)

5. A receptacle as claimed in claim 1, wherein the securing means is a press-fit arrangement.

6. A receptacle as claimed in claim 1, wherein the securing means is a self-securing arrangement.

7. A receptacle according to claim 6, wherein the receptacle body is self-securing to the collection bag.

8. (canceled)

9. (canceled)

10. (canceled)

11. A receptacle according to claim 1, wherein the receptacle body is self-securing to the collection bag at a plurality of positions.

12. (canceled)

13. (canceled)

14. A receptacle according to claim 1, wherein the receptacle is in the form of a container for holding the bodily waste materials received upon discharge of the collection bag.

15. (canceled)

16. (canceled)

17. A receptacle according to claim 1, wherein the receptacle is toilet-flushable.

18. A receptacle according to claim 1, wherein the receptacle is collapsible from an expanded state for use to receive the discharged waste materials to a collapsed state for storage.

19. A receptacle according to claim 18, wherein the collapsed state of the receptacle is a substantially flat condition thereof.

20. A receptacle according to claim 1, wherein the elongate body of the receptacle becomes narrower in a direction from the top end thereof towards the base end thereof.

21. A receptacle according to claim 1, further comprising a closure for its inlet opening.

22. A receptacle according to claim 1, further comprising at least one finger grip portion which projects away from the receptacle body about the inlet opening.

23. A receptacle according to claim 1, further comprising a guard portion on the receptacle which is attached to the receptacle adjacent the inlet opening and which is arranged to project from the receptacle to a position alongside the collection bag.

24. A receptacle according to claim 1, comprising a connection portion on the receptacle for connection to the collection bag.

25. A receptacle according to claim 24, wherein the connection portion takes the form of a wrap around which is for wrapping about and connecting to the collection bag.

26. A receptacle as claimed in claim 25, wherein the connection portion is a separate piece.

27. A receptacle as claimed in claim 24, wherein the connection portion attaches to the container portion of the receptacle and to the collection bag.

28. A receptacle as claimed in claim 24, wherein the connection portion has adhesive attachment points.

29. A receptacle as claimed in claim 24, wherein the connection portion is selected from the group comprising: ‘L’ shaped, “I” shaped and “T” shaped portions.

30. (canceled)

31. A method for disposing of bodily waste materials comprising the steps of:
(i) attaching a receptacle to a collection bag, so that the inlet opening receives the bodily waste discharge outlet of the collection bag;
(ii) discharging the bodily waste materials into the receptacle; and
(iii) optionally disposing of the receptacle and any contents thereof.

32. (canceled)

33. A method according to claim 31, wherein the receptacle is disposed of by flushing down a toilet.

34. A kit comprising:
a) an elongate body with a top end, a base end, an intermediate portion between the top end and the base end, and formed with an internal, bodily waste material-receiving chamber; an inlet opening formed on the body, proximate to, or at, the top end thereof, the body being adapted to receive within the inlet opening the bodily waste discharge outlet of the collection bag so as to allow bodily waste materials within the collection bag to be discharged into the bodily waste material-receiving chamber; and
securing means for holding the bodily waste material-receiving chamber in position for discharge of the bodily waste materials into the bodily waste material-receiving chamber from the collection bag.

35. A receptacle according to claim 1, wherein the receptacle is constructed of a biodegradable plastic.

36. A receptacle according to claim 1, wherein the receptacle is constructed of a polyvinyl film including a polyvinyl alcohol film.
